(19) United States
(12) Patent Application Publication
(10) Pub. No.: US 2015/0223908 A1
(43) Pub. Date: Aug. 13, 2015

(54) System and Method For Simultaneous Deployment of a Plurality of Fiducials

(71) Applicants: Jody Westerfeld, Greensburg, IN (US); David Rezac, Westborough, MA (US)

(72) Inventors: Jody Westerfeld, Greensburg, IN (US); David Rezac, Westborough, MA (US)

(21) Appl. No.: 14/502,650

(22) Filed: Sep. 30, 2014

Related U.S. Application Data
(60) Provisional application No. 61/939,297, filed on Feb. 13, 2014.

(51) Int. Cl. A61B 19/00; A61M 5/315

(52) U.S. Cl. CPC A61B 19/54; A61M 5/31571; A61B 2019/5487

(57) ABSTRACT

Provided is a system and method for simultaneous deployment of a plurality of fiducials. Various example embodiments may include an elongated hollow tube having an interior surrounding a distal portion of each of a plurality of fiducial carriers each having a fiducial removably attached therewith. Various example embodiments may include a spreading mechanism adapted to spread the distal ends of the plurality of fiducial carriers when the distal ends of the plurality of fiducial carriers are simultaneously extended beyond the distal end of the elongated hollow tube. Also provided in various example embodiments is an ejecting mechanism adapted to eject and implant the plurality of fiducials simultaneously when the fiducials are extended beyond the distal end of the elongated hollow tube and separated by a distance.

### CROSS-REFERENCE TO RELATED APPLICATIONS

This application claims priority to and incorporates herein by reference U.S. Provisional Application No. 61/939,297, filed on Feb. 13, 2014.

### FEDERALLY SPONSORED RESEARCH OR DEVELOPMENT

None.

### TECHNICAL FIELD

The present invention relates generally to medical devices and systems, and more particularly to fiducial deployment systems and methods.

### BACKGROUND

Medical procedures often require locating and treating target areas within a patient. Focused, dose-delivery radiation therapy requires locating the target with a high degree of precision to limit damaging healthy tissue around the target. It is particularly important to know or estimate the precise location of the target in radiation oncology because it is desirable to limit the exposure of adjacent body parts to the radiation in a patient already suffering the depredations of cancer. However, in all treatment procedures, whether radiologic or otherwise, it is most desirable to be able to accurately target a region to be treated. For example, the clinical success of focused, dose-delivery procedures, such as Stereotactic Body Radiation Therapy (SBRT), is based on the accuracy of target identification and precise patient positioning.

In many applications, it is not possible to directly view a treatment target or portion thereof (such as, for example, a cancerous tumor, cyst, pseudocyst, or other target) that needs to be acted on in some manner. As one example, when treating a lung or pancreatic tumor with radiation, it may not be possible to view the actual tumor within the patient immediately before the radiation treatment. It is therefore highly advantageous to have some mechanism for permitting the tumor to be located accurately so that the radiation treatment can be targeted at the tumor while avoiding damage to healthy tissue.

Even for target regions that may be visualized using CAT (computer-assisted tomography) scans, MRI (magnetic resonance imaging), x-rays, ultrasound, or other techniques, difficulties often arise in targeting a treatment. This is particularly true for target regions within a torso of a patient and soft tissue regions. Due to the mobility of tissues in those regions (e.g., movement of internal organs during respiration and/or digestion, the movement of breast tissue with any change of body position), a target region may not remain fixed relative to anatomical landmarks and/or to marks that can be placed onto an external surface of a patient's body during one of those visualization procedures.

Several techniques have been developed to address this problem. One such technique is to place markers into the patient along the margins of the target region. The markers may be active (e.g., emitting some kind of signal useful in targeting a therapy) or passive (e.g., non-ferromagnetic gold) markers called fiducials that can be used for targeting under ultrasound, MRI, x-ray, or other targeting techniques, which may be included in a treatment device).

A fiducial is typically formed of a radio-opaque material so that the target can be effectively located and treated with a device that targets a site using the fiducials as positional markers under radiographic detection. Typically, the fiducials may be inserted into the patient during a simple operation. Percutaneous placement is most commonly used. However, use of minimally-invasive placement via an endoscope has recently developed for fiducial placement into a patient's internal organs. For example, percutaneous placement of fiducials along the margins of a pancreatic tumor can be complex and painful (particularly for obese patients, where the needle size is necessarily larger). Another process using percutaneously implanted objects in a patient is brachytherapy. In brachytherapy, radioactive sources or “seeds” are implanted into and/or adjacent a tumor to provide a high dose of radiation to the tumor, but not the healthy tissue surrounding the tumor.

FIGS. 1A and 1B show longitudinal sectional views of a two-piece introducer 100 of the prior art useful for placement of brachytherapy seeds or fiducials. Referring first to FIG. 1A, the introducer 100 includes a needle 102 and a stylet 104 slidably disposed within the needle 102. The stylet 104 includes a first handle 101 and a blunt distal end 106. The needle 102 includes a second handle 103 and a bevel-tipped cannula 108 extending through the second handle 103. The cannula 108 is configured to hold a seed/fiducial 110. The cannula 108 has a distal tip 105 configured for percutaneous implantation of the seed/fiducial 110 into the patient.

In a “pre-loaded configuration,” the seed/fiducial 110 is retained in the cannula 108 by a plug 112 made from bone wax or other suitable bio-compatible material(s). This is typically accomplished by a “muzzle-loading” technique where the fiducial is placed into the distal end of the needle and then held in place by the bone wax plug. This can present some challenges, as the bone wax plug 112 can be visible as an artifact in the patient, potentially interfering with clear visualization of body structures or treatment devices. With this configuration, the cannula 108 must be withdrawn and reloaded after delivery of each seed/fiducial 110. If the target locations for the fiducials are very far apart, use of a single percutaneous introducer cannula/trocar for multiple introductions of the cannula 108 may not be possible. In such a circumstance, the patient must endure several percutaneous punctures (and the increased attendant risk of infection for each). Multiple percutaneous punctures can also be problematic, especially to the lung as each puncture significantly increases the risk for pneumothoraces that require chest tube placement, lengthened hospital stay, and other unwanted adverse results.

To implant the desired arrangement of seeds/fiducials 110 at a target location in a patient, an operator pushes the cannula 108 in a first direction (arrow A) to insert the tip 105 into the patient (typically under fluoroscopic visualization). The operator then pushes the second handle 103 further in the first direction to position the tip 105 at the desired depth within the patient where a seed/fiducial 110 is to be implanted. Throughout this motion, the operator moves the needle 102 and the stylet 104 together as a unit. At the desired depth/location, the operator grasps the first handle 101 with one hand and the second handle 103 with the other hand. Then, the operator holds the first handle 101 stationary while simultaneously sliding the second handle 103 back in a second direction (arrow B) toward the first handle 101. As shown in FIG. 1B, this movement causes the cannula 108 to retract over the seed/fiducial 110 to implant it in the patient. Alternatively, the operator may move the first handle 101 in the first direction (arrow A) while sliding the second handle 103 back in the second direction (arrow B) or holding it stationary. This causes the stylet 104 to push the seeds 110 out of the cannula 108. The procedure is then repeated to place other seeds/fiducials 110. When being used for targeting of radiation therapy, a minimum of three fiducials are typically required. However, other numbers may be used, such as two, four, or any suitable number of fiducials.

As will be appreciated from the disclosed structure, after deploying one fiducial, one may alternatively reload the introducer 100 from the proximal end by completely withdrawing the stylet 104, then placing another fiducial into the needle lumen and advancing it there through to a second location to which the distal needle tip 105 has been directed (a “breech-loading” technique). Provided that the fiducial target sites are sufficiently close together to allow this technique, it can reduce the number of percutaneous punctures or other access procedures needed to place more than one fiducial. However, it creates a problem for procedures where ultrasound is being used or is to be used in the near-future because it introduces air pockets into the tissue and related fluids. Those air pockets with tissue and/or fluid are echogenic in a manner that can interfere with ultrasound visualization of a target area and/or tools being used to diagnose or treat in/around the area. In some brachytherapy techniques, a series of fiducials may be preloaded into the needle—either separately or connected by a suture or similar device then placed together in fairly close proximity; however, such a technique typically is not effective for placing three or more fiducials in sufficiently disparate locations to use for targeting a treatment relative to, for example, margins of a tumor.

The process is similar when implemented endoscopically in the manner developed rather recently, except that the needle and stylet are of the type known in the art for use through the working channel of an endoscope. One limitation of current endoscopic techniques is the size of fiducial that can be introduced. With the size limitation of endoscope working channels, the largest needle that can typically be used without risking bending, crimping, curving or otherwise damaging a needle (that does not have an internal stylet or other support) during advancement out of the endoscope to an anatomical target is a 19-gauge needle. This limits the size of the fiducial that can be introduced through the needle lumen using current, cylindrical fiducials. The endoscopic technique generally suffers from the same reloading problems as described above. Even though the external percutaneous punctures are not an issue, having to withdraw and reload takes up valuable time and complicates the procedure, potentially requiring additional personnel, whether only the stylet is withdrawn for “breech-loading” or the entire device is withdrawn for “muzzle-loading.”

In connection with using ultrasound and particularly endoscopic ultrasound (EUS) for navigation and placement of fiducials, it has been proposed to provide multiple fiducials in a needle that can be introduced in a controlled serial manner (one at a time) rather than requiring manual reloading after placement of each fiducial. Regarding that idea, incorporated herein by reference is U.S. patent application Publication No. US20130096427 A1, application No. US 13/645,614, published on Apr. 18, 2013, filed on Oct. 5, 2012, by Mark Murray, Triona Campbell, Vincent McHugo, Michael Clancy, and John Neilan, and originally assigned to Cook Medical Technologies LLC (“the '427 Publication”). In fact, the above background description is largely copied from the '427 Publication, as permitted and encouraged under U.S. copyright and patent law.

The '427 Publication is not the only art to suggest providing multiple fiducials in a needle that can be introduced in a controlled serial manner (one at a time) rather than requiring manual reloading after placement of each fiducial. Regarding that and other matters, the following additional reference is also incorporated herein by reference: U.S. patent application Publication No. US20130197356 A1, application Ser. No. US 13/751,752, published on Aug. 1, 2013, filed on Jan. 28, 2013, by Michael S. Clancy and John Neilan, and originally assigned to Cook Medical Technologies Inc. Also incorporated herein by reference is U.S. patent application Publication No. US20130006101 A1, application Ser. No. US 13/526,991, published on Jan. 3, 2013, filed on Jun. 19, 2012, by Vincent McHugo, Michael Clancy, and John Neilan. Additionally incorporated herein by reference is U.S. patent application Publication No. US20100280367 A1, application Ser. No. US 12/764,432, published on Nov. 4, 2010, filed on Apr. 21, 2010, by Richard W. Ducharme and Vihar C. Surti. Further incorporated herein by reference is U.S. patent application Publication No. US20090105584 A1, application Ser. No. US 12/211,436, published on Apr. 23, 2009, filed on Sep. 16, 2008, by Andrew Jones, and originally assigned to Civco Medical Instruments Co., Inc. Incorporated also by reference herein is U.S. patent application Publication No. US20100268014 A1, application Ser. No. US 12/427,667, published on Oct. 21, 2010, filed on Apr. 21, 2009, by Charlie Pitman, and originally assigned to Core Oncology, Inc.

While providing multiple fiducials in a needle that can be introduced in a controlled serial manner (one at a time) can be an improvement over requiring manual reloading after placement of each fiducial, deploying the fiducials one at a time around the targeted area can require re-navigating items such as a scope and catheter for each fiducial, which can be time consuming and increases chances of errant placement (e.g., placement not in accordance to Stereotactic Body Radiation Therapy (SBRT) positioning guidelines, jamming, or other malfunctions and issues). Further, having to re-navigate and steer fiducials into the desired positions multiple times in one setting may also negatively affect the patient by increasing the time the patient receives narcotics or paralytics while under sedation.

### SUMMARY

The present invention elegantly addresses all the above challenges and provides numerous additional benefits. In various example embodiments the solution discovered by the present inventor may comprise a system and method for deployment of a plurality of fiducials simultaneously, examples of which are shown and described. For example, provided in various example embodiments is an apparatus for simultaneous deployment of a plurality of fiducials, which may comprise: an elongated hollow tube adapted to be inserted into a patient such that a distal end of the elongated hollow tube is placed proximate a user determined location in the patient; the elongated hollow tube having an interior surrounding a distal portion of each of a plurality of fiducial carriers, each of the plurality of fiducial carriers having a fiducial removably attached therewith near a distal end of the fiducial carrier; a spreading mechanism adapted to spread the distal ends of the plurality of fiducial carriers, such that at least one of the fiducials is separated from an adjacent fiducial by a fiducial separation distance, when the distal ends of the plurality of fiducial carriers are simultaneously extended beyond the distal end of the elongated hollow tube; and an ejecting mechanism adapted to eject and implant the plurality of fiducials simultaneously when the fiducials are extended beyond the distal end of the elongated hollow tube and separated by the fiducial separation distance. In various example embodiments the spreading mechanism includes the distal ends of the plurality of fiducial carriers being biased to urge away from each other as they are extended beyond the distal end of the elongated hollow tube. In various example embodiments the apparatus is further adapted to extend the fiducial carriers beyond the distal end of the elongated hollow tube without ejecting the plurality of fiducials. In various example embodiments each of the fiducial carriers further comprises a fiducial positioner adapted to enable user positioning of the distal end of the fiducial carrier prior to ejecting the plurality of fiducials. In various example embodiments the fiducial positioner comprises needle tips at distal ends of the fiducial carriers. In various example embodiments each of the plurality of the fiducial carriers comprises: an elongated cylinder having a needle tip at a distal end; the elongated cylinder adapted to contain until ejected, proximate its distal end, at least one fiducial; and a pusher at least partially contained in the fiducial carrier, the pusher adapted to eject the fiducial from the fiducial carrier. In various example embodiments the ejecting mechanism comprises a pusher handle connected to proximal ends of the pushers. In various example embodiments the apparatus is adapted to cause the fiducials to separate from each other by one or more predetermined fiducial separation distances when a hub is moved longitudinally one or more predetermined hub distances. In various example embodiments the plurality of fiducial carriers comprises three fiducial carriers. In various example embodiments the apparatus further includes a safety mechanism adapted to prevent unintentional ejection of the fiducials.

Also provided in various example embodiments is a method of simultaneously deploying a plurality of fiducials, which may comprise the steps of: inserting a distal end of an elongated hollow tube to a location in a patient such that a distal end of the elongated hollow tube is placed proximate a user determined location in the patient; said elongated hollow tube having an interior adapted to surround a distal portion of each of a plurality of fiducial carriers, each of the plurality of fiducial carriers having a fiducial removably attached therewith near a distal end of the fiducial carrier; simultaneously extending a distal end of each of the plurality of fiducial carriers beyond the distal end of the elongated hollow tube; spreading the distal ends of the plurality of fiducial carriers, such that at least one of the fiducials is separated from an adjacent fiducial by a predetermined fiducial separation distance; and expelling and implanting the plurality of fiducials simultaneously when the fiducials carriers are extended and the fiducials are separated by the predetermined fiducial separation distance. In various example embodiments the distal ends of the plurality of fiducial carriers are biased to urge away from each other as they are extended beyond the distal end of the elongated hollow tube. In various example embodiments the method further includes the step of positioning the distal ends of the plurality of fiducial carriers prior to ejecting the plurality of fiducials. In various example embodiments the steps of expelling and implanting the plurality of fiducials simultaneously when the fiducials are separated comprises simultaneously pushing a plurality of pushers, each of which is at least partially contained in each of the plurality of fiducial carriers and is adapted to eject a fiducial from a corresponding fiducial carrier. In various example embodiments the step of simultaneously pushing a plurality of pushers comprises pushing a handle connected to a proximal end of the pushers. In various example embodiments the method further includes the step of: simultaneously expelling and implanting the plurality of fiducials by pushing a hub distally one or more predetermined hub distances and thereby causing the fiducials to separate to one or more predetermined fiducial separation distances. In various example embodiments the method further includes the step of operating a safety mechanism adapted to avoid premature ejection of fiducials.

Also provided is a medical targeting system comprising an apparatus adapted to simultaneously deploy a plurality of fiducials. In various example embodiments the system may comprise an elongated hollow tube adapted to be inserted into a patient such that a distal end of the elongated hollow tube is placed proximate a user determined location in the patient; the elongated hollow tube having an interior adapted to surround a distal portion of each of a plurality of fiducial carriers, each of the plurality of fiducial carriers having a fiducial removably attached therewith near a distal end of the fiducial carrier; a spreading mechanism adapted to spread the distal ends of the plurality of fiducial carriers, such that at least one of the fiducials is separated from an adjacent fiducial by a fiducial separation distance, when the distal ends of the plurality of fiducial carriers are extended beyond the distal end of the elongated hollow tube; and an ejecting mechanism adapted to enable a user to eject and implant the plurality of fiducials simultaneously when the plurality of fiducial carriers are extended beyond the distal end of the elongated hollow tube and the fiducials are separated. In various example embodiments each fiducial carrier further comprises a fiducial positioner adapted to enable a user to position the distal end of the fiducial carrier prior to ejecting the plurality of fiducials.

Example embodiments are shown and described in this patent application in the accompanying written description and figures. Additional aspects, alternatives and variations as would be apparent to persons of skill in the art are also disclosed herein and are specifically contemplated as included as part of the invention. The invention is set forth only in the claims as allowed by the patent office in this or related applications, and the foregoing summary descriptions of certain examples are not in any way to limit, define or otherwise establish the scope of legal protection.

### BRIEF DESCRIPTION OF THE DRAWINGS

The accompanying figures illustrate certain aspects of example embodiments of the invention.

FIG. 1A is a diagram depicting examples of known systems as described in the Background.

FIG. 1B is a diagram depicting examples of known systems as described in the Background.

FIG. 2 is a perspective view of one embodiment of an example system for simultaneous deployment of a plurality of fiducials, shown undeployed.

FIG. 3a is a perspective view of the example system of FIG. 2, shown simultaneously deploying a plurality of spread-apart fingers containing fiducials.

FIG. 3b is an enlarged, partial cutaway view of FIG. 3a, showing example internal features of the deployed fingers.

FIG. 3c is a section view of FIG. 3b along line C-C, showing example internal features.

FIG. 4a is a perspective view of the example system of FIGS. 2 and 3a, shown simultaneously deploying a plurality of fiducials from the ends of the spread-apart fingers.

FIG. 4b is an enlarged, partial cutaway view of FIG. 4a, showing simultaneously deployment of a plurality of a fiducial from the end of each of the spread-apart fingers.

FIG. 5 is a partially transparent perspective view of proximal portions of the example system of FIGS. 2, 3a, and 4a.

FIG. 6 is a partially-exploded perspective view of the example system of FIGS. 2, 3a, and 4a, shown simultaneously deploying a plurality of spread-apart fingers containing fiducials.

FIG. 7 is an exploded perspective view of the example system of FIGS. 2, 3a, 4a, and 6, showing example internal components for deploying a single fiducial, which may be duplicated any suitable number of times in other example embodiments.

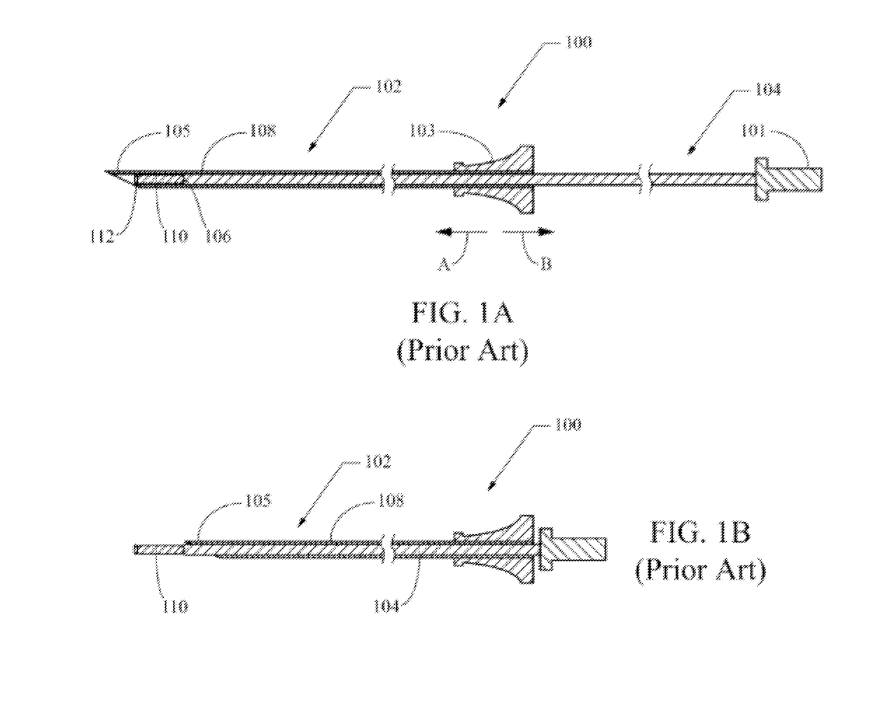

### DETAILED DESCRIPTION OF EXAMPLE EMBODIMENTS

Reference is made herein to some specific examples of the present invention, including any best modes contemplated by the inventor for carrying out the invention. Examples of these specific embodiments are illustrated in the accompanying figures. While the invention is described in conjunction with these specific embodiments, it will be understood that it is not intended to limit the invention to the described or illustrated embodiments. To the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, process operations well known to persons of skill in the art have not been described in detail in order not to obscure unnecessarily the present invention. Various techniques and mechanisms of the present invention will sometimes be described in singular form for clarity. However, it should be noted that some embodiments include multiple iterations of a technique or multiple mechanisms unless noted otherwise. Similarly, various steps of the methods shown and described herein are not necessarily performed in the order indicated, or performed at all in certain embodiments. Accordingly, some implementations of the methods discussed herein may include more or fewer steps than those shown or described. Further, the techniques and mechanisms of the present invention will sometimes describe a connection, relationship or communication between two or more entities. It should be noted that a connection or relationship between entities does not necessarily mean a direct, unimpeded connection, as a variety of other entities or processes may reside or occur between any two entities. Consequently, an indicated connection does not necessarily mean a direct, unimpeded connection unless otherwise noted.

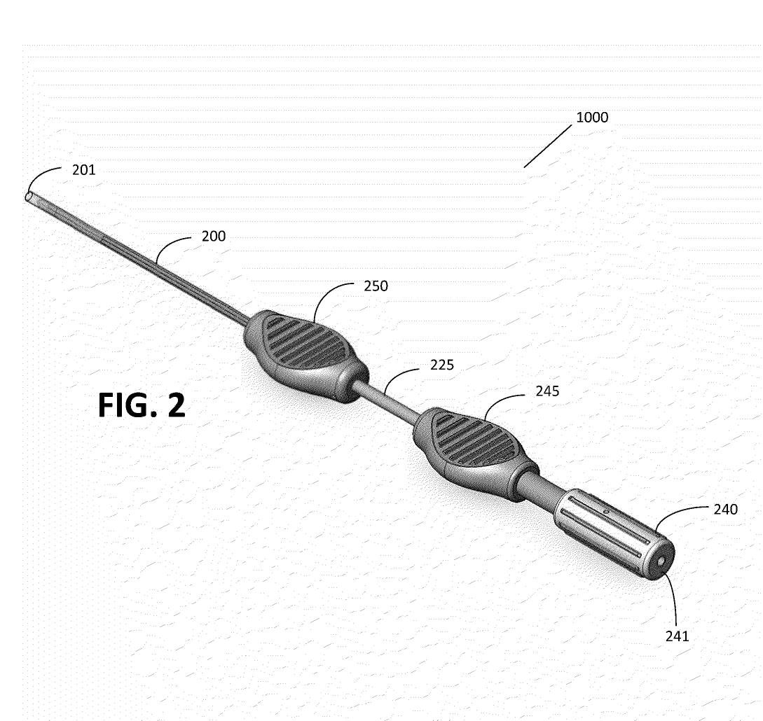

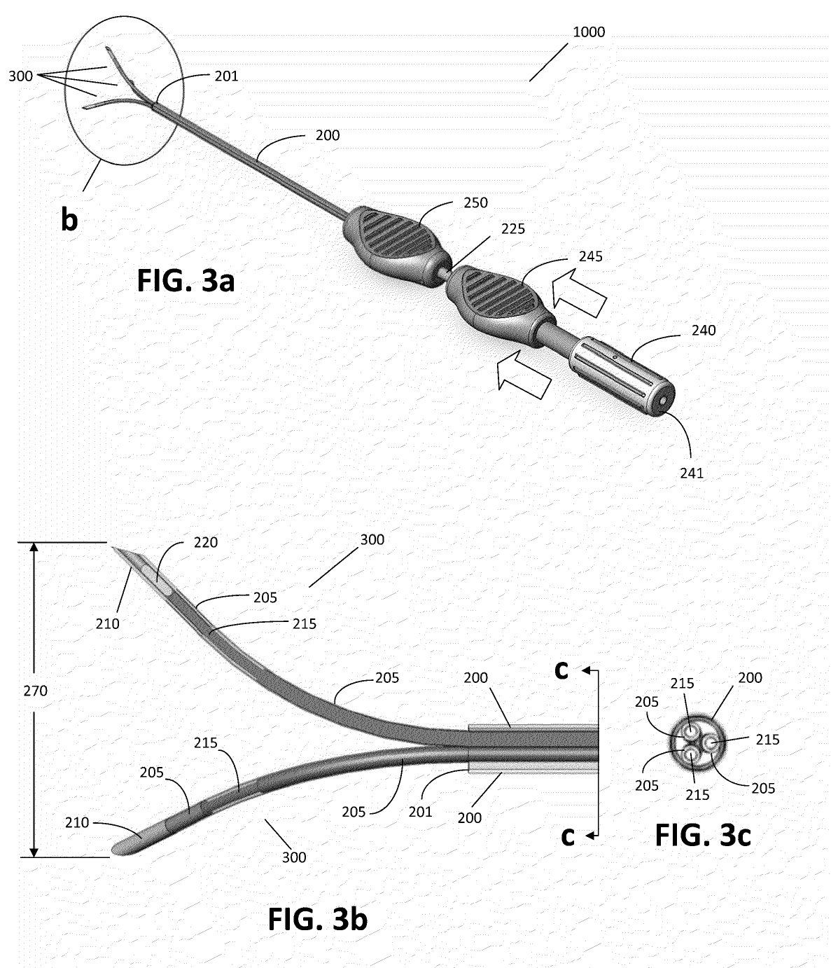

The present device or system 1000 can implant a plurality of fiducials 220 in one movement, decreasing time, increasing accuracy, and avoiding the problems inherent in serial placement, such as patient movement and pneumothoraces between placements. With reference to FIGS. 2 through 7, various example embodiments of the present system 1000 may comprise multiple fingers 300, each having a fiducial 220 removably attached therewith, which may extend distally away from a distal end 201 of a catheter 200 upon activation by a user, the fingers 300 spreading apart and moving radially away from each other as they extend distally outward away from distal end 201, for instance by a predetermined amount or distance 270, as shown in FIG. 3b (fingers 300 also referred to as elongated cylinders or fiducial carriers in certain embodiments).

A user such as a physician may receive an undeployed system 1000 ready to use, for instance as shown in FIG. 2. An activation system which may comprise a fiducial positioner may be provided at or near a proximal end 241 of the system 1000, and a catheter 200 may extend distally from the activation system. The activation system may be adapted to cause a plurality of fiducials to simultaneously deploy from the distal end 201 of the catheter 200. In the non-limiting example embodiment shown in the figures, the activation system may comprise a proximal shaft hub 245 adapted to be gripped by a user and further adapted to translate longitudinally relative to a distal shaft hub 250, which itself may be adapted to be gripped by a user. In the example embodiment shown in the figures, translating the proximal shaft hub 245 longitudinally toward the distal shaft hub 250 may cause a plurality of fingers 300 to deploy from the end of the catheter 200, for instance as shown in FIG. 3a, for instance to act as a fiducial positioner. The fingers 300 may be adapted by any suitable spreading mechanism to tend to spread away from each other as they extend distally away from the distal end 201 of the catheter 200. This tendency or bias may be accomplished by any suitable spreading mechanism, such as by preloading or biasing the fingers 300 with a spring tension urging them radially outward from the centerline of the catheter 200, such that the spring tension tends to be relieved as the fingers 300 emerge from the end of the catheter 200 and bend radially outward away from the centerline of the catheter, and thus radially away from each other. For example, the needle actuation sheaths 205 or the pushers 215 or both may be pre-formed with curves bending radially outward from the centerline of the catheter 200, which curves are elastically deformed into nominally straight lines parallel with the centerline of the catheter 200 when the needle actuation sheaths 205 and the pushers 215 are in the retracted position within the catheter 200, for instance as shown in FIG. 2. Alternatively the fingers 300 may be urged away from each other as they extend away from the distal end 201 of the catheter 200 by any type of mechanical guidance mechanism, such as one or more wedges (not shown), that would tend to bend or direct the fingers 300 radially away from each other as they exit the distal end 201 of the catheter. Any other suitable spreading mechanism may be used. In certain example embodiments a predetermined longitudinal movement of the proximal shaft hub 245 one or more hub distances may cause the fingers 300 to extend and spread apart by approximately a predetermined distance, such as distance 270 shown in FIG. 3b.

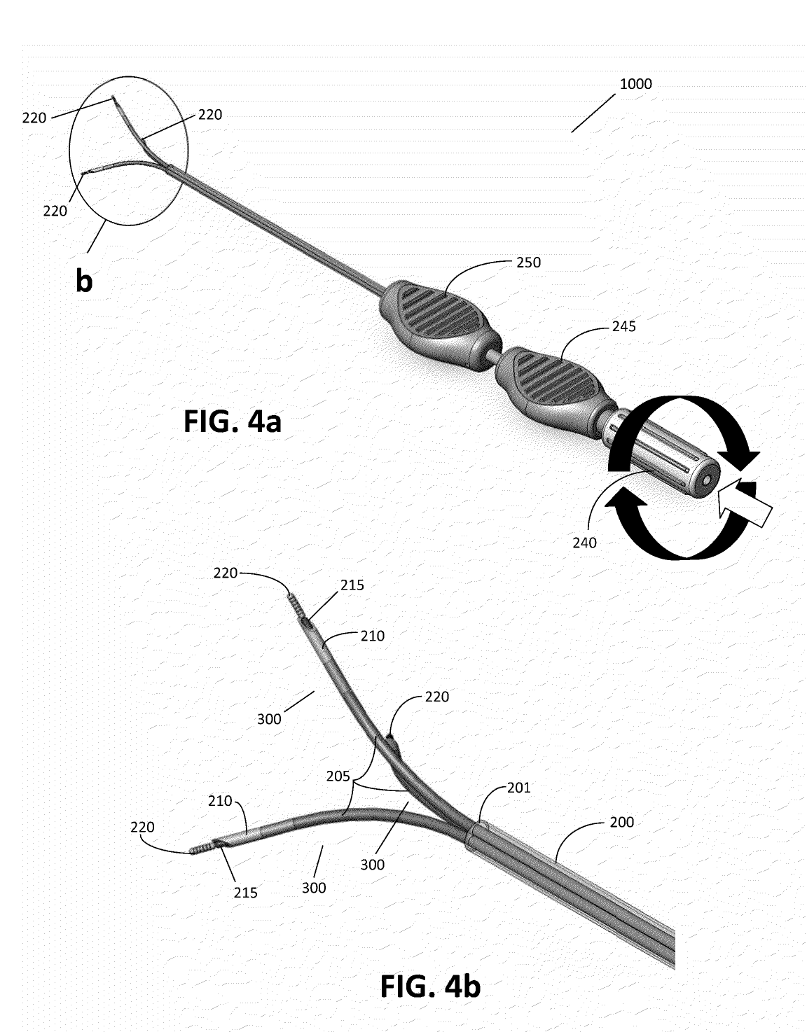

In the non-limiting example embodiment shown in the figures, the activation system may further comprise a proximal cap 240 adapted to be gripped by a user and rotated about the centerline of the system 1000, while also being longitudinally translated from the proximal end 241 toward the distal end 201 of the system 1000, for instance toward the proximal shaft hub 245 as shown in FIG. 4a. In the non-limiting example embodiment shown in the figures, rotating the proximal cap 240 about the centerline of the system 1000, while also longitudinally translating it toward the proximal shaft hub 245 causes the plurality of fiducials 220 to simultaneously deploy from their corresponding ends of the fingers 300, as shown in FIGS. 4a and 4b, and thereby simultaneously implant at the desired locations within a patient's body (not shown).

It will be understood by those of skill in the art that entirely different activation systems may be employed that would function equivalently to simultaneously deploy any suitable number of fiducials, and the invention is not limited to any particular activation or deployment system unless specifically claimed. In the non-limiting example embodiment shown in FIGS. 2-7, the activation system causes simultaneous deployment of fingers 300 then fiducials 220 by an example mechanism comprising the following example components.

Provided in various example embodiments is an outer sheath 200 (also referred to as an elongated hollow tube), which may be a catheter or cannula. Outer sheath 200 may be formed from any suitable material, such as, for example, Nylon, Pebax, Urethane, Polyimide, PET, other common engineering plastic, possibly reinforced. Outer sheath 200 may be attached and assembled with distal shaft hub 250 using any suitable means, such as, for example, adhesive bonding, set screw, or welding.

Provided in various example embodiments is a needle actuation sheath 205 (also referred to as an elongated cylinder or fiducial carrier in certain embodiments), surrounded in a distal portion by outer sheath 200 when in its pre-deployment mode, and comprising the outer surface of fingers 300 when deployed, for instance as shown in FIGS. 3b and 3c. Needle actuation sheath 205 may be formed from any suitable material, such as, for example, Polyimide, PET, Nylon, PEEK, possibly reinforced (such as braided). A plurality of needle actuation sheaths 205 (one for each fiducial 220) may be attached and assembled with proximal shaft hub 245 using any suitable means, such as, for example, adhesive bonding, overmolded in place, or compression fitting(s).

Provided in various example embodiments are one or more needle tips 210 (one for each fiducial 220), to facilitate implantation of the fiducials 220 in the patient's tissue (not shown). Needle tips 210 (also referred to as an elongated cylinder or fiducial carrier in certain embodiments) may be formed from any suitable material, such as, for example, Stainless Steel or Nidinol. Needle tips 210 may be attached and assembled with needle actuation sheaths 205 using any suitable means, such as, for example, adhesive bonding, overmolded in place, or external clamp.

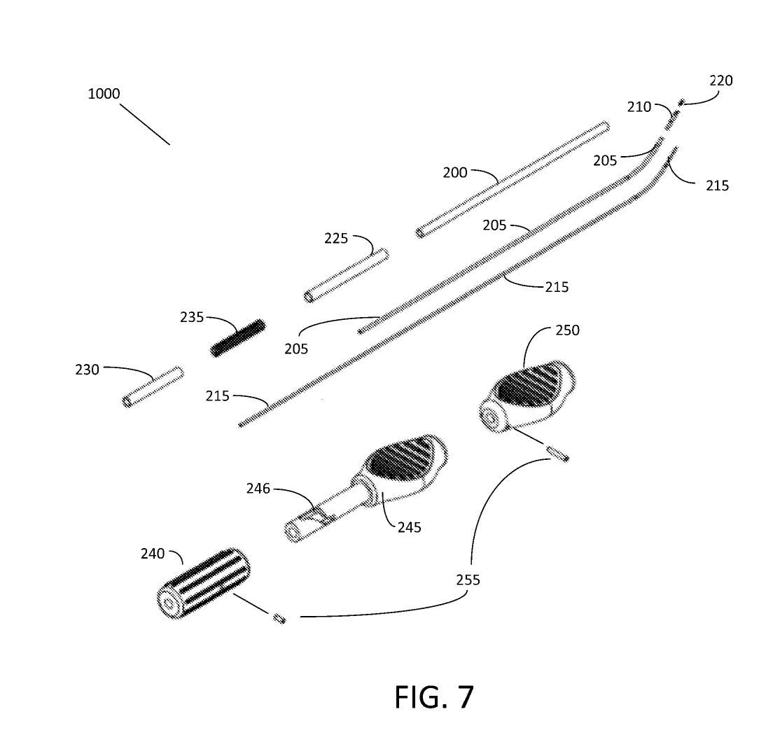

Provided in various example embodiments are one or more pushers 215 (which may be a stylet) largely surrounded by needle actuation sheath 205. Each pusher 215 is adapted to translate longitudinally within each corresponding needle actuation sheath 205, and is adapted to communicate the distal motion of the proximal shaft hub 245 to each fiducial 220. The exploded view in FIG. 7 shows one needle actuation sheath 205 and one pusher 215, recognizing that any suitable number of these parts may be used in a single system 1000, depending on dimensional constraints of various components and the patient's body, and the number of fiducials 220 that are to be deployed simultaneously (for instance, 2, 3, 4, 5, 6, or more). Pushers 215 may be formed from any suitable material, such as, for example, Nidinol or Spring Steel. Pushers 215 may be attached and assembled with the proximal shaft hub 245 using any suitable means, such as, for example, adhesive bonding, set screw, or welding.

Provided in various example embodiments are one or more fiducial markers 220. Fiducial markers 220 may be formed from any suitable material, such as, for example, Gold, Platinum-Indium, Tantalum. Fiducial markers 220 may be removably attached and assembled within the needle tip 210 or needle actuation sheath 205 using any suitable means, such as, for example, a light press fit. Additionally or alternatively, plugs (not shown) may be placed in the distal portions of the needle tips 210 to retain the fiducials 220.

Provided in various example embodiments is a distal support tube 225 on which the proximal shaft hub 245 may longitudinally translate. Distal support tube 225 may be formed from any suitable material, such as, for example, Stainless Steel, Polycarbonate, or ABS. Distal support tube 225 may be attached and assembled with the distal shaft hub 250 using any suitable means, such as, for example, adhesive bonding, set screw, press fit, or overmolded in place.

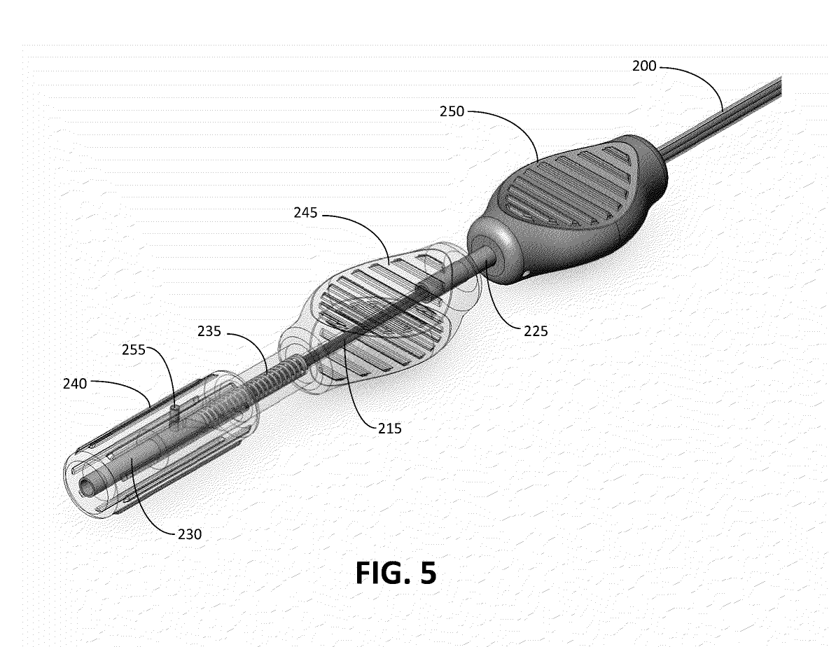

Provided in various example embodiments is a proximal support tube 230, as shown in FIGS. 5-7. Proximal support tube 230 is adapted to translate the longitudinal motion of the proximal cap 240 to the pushers 215 and thus to the fiducials 220. Proximal support tube 230 may be formed from any suitable material, such as, for example, Stainless Steel, Polycarbonate, or ABS. Proximal support tube 230 may be attached and assembled with the proximal cap 240 using any suitable means, such as, for example, adhesive bonding, set screw, press fit, or overmolded in place.

Provided in various example embodiments is a proximal cap return spring 235, adapted to urge the proximal cap 240 in a proximal direction, so that a user must intentionally push against the force of spring 235 in order to deploy the fiducials 220. Proximal cap return spring 235 may be formed from any suitable material, such as, for example, Stainless Steel or Nidinol. Proximal cap return spring 235 may be attached and assembled using any suitable means, such as, for example, being physically captured inside the pocket created by the assembly, for instance as shown in FIG. 5.

Provided in various example embodiments is a proximal cap 240 (also referred to as a pusher handle). Proximal cap 240 may be formed from any suitable material, such as, for example, HDPE, Nylon, Polycarbonate, Urethane, or ABS. Proximal cap 240 may be attached and assembled with proximal support tube 230 using any suitable means, such as, for example, adhesive bonding, set screw, press fit, or overmolded in place.

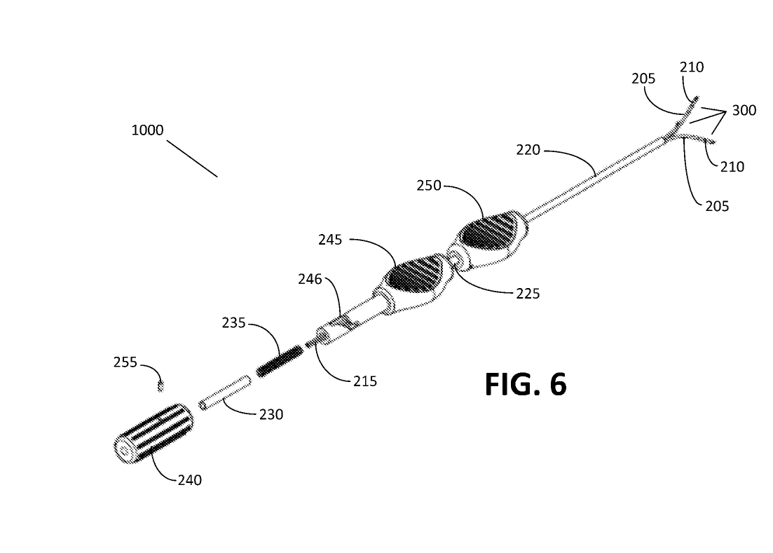

Provided in various example embodiments is a proximal shaft hub 245. Proximal shaft hub 245 may be adapted to slide longitudinally over distal support tube 225 and to distally push needle actuation sheaths 205 to extend the plurality of fingers 300 (which fingers 300 each comprise a needle actuation sheath 205 and needle tip 210 surrounding a movable pusher 215 and deployable fiducial 220). Proximal shaft hub 245 may be provided with a keyway 246 to control and guide the rotational and longitudinal motion of a key or similar device or protrusion 255 connected with the proximal cap 240, for instance as shown in FIGS. 5-6. Proximal shaft hub 245 may be formed from any suitable material, such as, for example, HDPE, Nylon, Polycarbonate, Urethane, or ABS. Proximal shaft hub 245 may be attached and assembled with needle actuation sheaths 205 using any suitable means, such as, for example, adhesive bonding, set screw, press fit, or overmolded in place.

Provided in various example embodiments is a distal shaft hub 250. Distal shaft hub 250 may be formed from any suitable material, such as, for example, HDPE, Nylon, Polycarbonate, Urethane, or ABS. Distal shaft hub 250 may be attached and assembled with outer sheath 200 (on a distal end), and with distal support tube 225 (on a proximal end) using any suitable means, such as, for example, adhesive bonding, set screw, press fit, or overmolded in place.

Provided in various example embodiments are one or more retaining pins 255. Retaining pins 255 may be formed from any suitable material, such as, for example, Stainless Steel or reinforced plastic. Retaining pins 255 may be attached and assembled as indicated in FIG. 7 using any suitable means, such as, for example, press fit, adhesive bonding, welding, or soldering. Alternatively, retaining pins 255 may comprise a set screw or any other suitable mechanical means. With respect to retaining pin 255 that extends into proximal cap 240, it may be adapted to act as a key to engage keyway 246 in proximal shaft hub 250. This would serve to guide and control the rotational and longitudinal movement of the proximal cap 240, so that a user must intentionally rotate and longitudinally translate the proximal cap 240 in a particular pattern while pushing against the force of spring 235, in order to deploy the fiducials 220. These features may combine to act as a safety mechanism adapted to prevent ejection of the fiducials 220 until the plurality of needle actuation sheaths or fiducial carriers 205 are extended beyond the distal end 201 of the elongated hollow tube or catheter 200 by a predetermined distance.

When appropriately positioned inside a patient's body, in the non-limiting example embodiment shown in the figures, a plurality of fingers 300, in this case three fingers 300, respectively extend distally outward from the distal end 201 and radially away from each other, for instance by a predetermined distance 270, when a physician or other user initially activates the device 1000 by gripping the distal shaft hub 245 and the proximal shaft hub 250, and translating the distal shaft hub 245 toward the proximal shaft hub 250, for instance by a predetermined distance that may correspond to the length of distal support tube 225. A physician or other user may then complete the activation of the device 1000 by gripping the proximal cap 240 and rotating it about the longitudinal axis of the device 1000 while moving it longitudinally from the proximal end 241 toward the distal end 201, as depicted in FIG. 4a. This will deploy the fiducials 220 as depicted in FIGS. 4a and 4b, for instance into the targeted tissue of a patient's body. The user may accurately position the needle tips 210 in the patient prior to simultaneously ejecting the plurality of fiducials 220.

A prong comprising multiple fingers 300 may be created by positioning an angle of 15-45 degrees between each finger 300 (measured in a plane perpendicular to the centerline of the distal end 201 of the catheter 200) and expelling each fiducial 220 a certain distance 270 apart, for instance 2 cm apart, meeting the standard SBRT fiducial marker placement guideline. Alternatively, two, three, or four fingers 300 may be positioned 180, 120, 90, or 60 degrees apart, or some prongs may be closer together than other prongs (for instance, two prongs could be 15 degrees apart, while a third prong is 172.5 degrees from each of the two prongs). Any suitable combination of number fingers 300 at any suitable relative orientations may be used.

After the fiducials 220 are simultaneously implanted (simultaneously meaning essentially simultaneously), the catheter system 1000 can then be removed and discarded from the lung and bronchoscope, leaving, a plurality of fiducial markers 220 in place for SBRT or other radio therapy, for example. Prior to removing the system 1000 from the patient's body, the user may retract the fingers 300 back inside the catheter or outer sheath 200 by longitudinally translating the distal shaft hub 245 proximally away from the proximal shaft hub 250 to return the distal end of the device 1000 to the position shown in FIG. 2.

This new apparatus, system, and method will allow physicians to implant a plurality of fiducials 220 essentially at once and in accordance to SBRT positioning or other guidelines, without having to re-navigate the scope and catheter as in present systems. This simultaneous system and method allows for a quicker procedure and will also be more precise and accurate because the multi-finger prong 300 may be designed to meet the standard SBRT fiducial marker placement guideline, for example. In certain example embodiments the system may be calibrated such that pushing the stylet forward one or more predetermined distances (via the example means disclosed herein or otherwise) will cause the placement of the fiducials to expand or open to one or more predetermined radii, diameters, shapes, orientations, and sizes.

It will be understood by those of skill in the art that in practice the system 1000 may be much longer than the system shown in the figures, specifically the catheter portion 200 and components therein may be much longer, as necessary to reach internal regions of a patient's body. The length of the system 1000 is not important to its description, however, so the system 1000 is shown shorter than usual for convenience of clear illustration.

Any of the suitable technologies set forth and incorporated herein may be used to implement various example aspects of the invention as would be apparent to one of skill in the art. Although exemplary embodiments and applications of the invention have been described herein including as described above and shown in the included example FIGS., there is no intention that the invention be limited to these exemplary embodiments and applications or to the manner in which the exemplary embodiments and applications operate or are described herein. Indeed, many variations and modifications to the exemplary embodiments are possible as would be apparent to a person of ordinary skill in the art. The invention may include any device, structure, method, or functionality, as long as the resulting device, system or method falls within the scope of one of the claims that are allowed by the patent office based on this or any related patent application.

1. A one-piece apparatus ready to be inserted into an endoscope and capable of simultaneous deployment of a plurality of fiducials, comprising:
an elongated hollow tube fixedly attached with a first grippable member and adapted to be inserted into a patient such that a distal end of the elongated hollow tube is placed proximate a user determined location in the patient;
the elongated hollow tube having an interior surrounding a distal portion of each of a plurality of fiducial carriers fixedly attached with a second grippable member longitudinally translatable relative to the first grippable member, each of the plurality of fiducial carriers having a fiducial removably attached therewith near a distal end of the fiducial carrier;
a spreading mechanism adapted to spread the distal ends of the plurality of fiducial carriers, such that at least one of the fiducials is separated from an adjacent fiducial by a fiducial separation distance, when the distal ends of the plurality of fiducial carriers are simultaneously extended beyond the distal end of the elongated hollow tube by longitudinally translating the second grippable member relative to the first grippable member by a predetermined distance; and
an ejecting mechanism adapted to eject and implant the plurality of fiducials simultaneously at a user-determined time after the fiducials are extended beyond the distal end of the elongated hollow tube and separated by the fiducial separation distance, the ejecting mechanism comprising a third grippable member longitudinally translatable and rotatable relative to both the first grippable member and the second grippable member, the third grippable member attached with a plurality of pushers that push the plurality of fiducials out of the fiducial carriers when the third grippable member is longitudinally translated and rotated in a predetermined path relative to both the first grippable member and the second grippable member.

2. The apparatus of claim 1, wherein the spreading mechanism includes the distal ends of the plurality of fiducial carriers being biased to urge away from each other as they are extended beyond the distal end of the elongated hollow tube.

3. The apparatus of claim 1, further comprising a spring pushing against the third grippable member, such that a user would have to overcome the force of the spring in order to longitudinally translate the third grippable member relative to both the first grippable member and the second grippable member.

4. The apparatus of claim 1, adapted to enable user-positioning of the distal end of the fiducial carrier prior to ejecting the plurality of fiducials by movement of the entire one-piece apparatus within an endoscope in a patient.

5. The apparatus of claim 1, comprising needle tips at distal ends of the fiducial carriers.

6. The apparatus of claim 1, wherein each of the plurality of pushers is at least partially contained in a corresponding one of the plurality of fiducial carriers, each of the plurality of fiducial carriers further comprising:
an elongated cylinder having a needle tip at a distal end; and
the elongated cylinder adapted to contain until ejected, proximate its distal end, at least one fiducial.

7. The apparatus of claim 1, wherein the movement of the third grippable member relative to both the first grippable member and the second grippable member is limited by a retaining pin that extends from the third grippable member into a keyway in the second grippable member.

8. The apparatus of claim 1, wherein the first grippable member comprises a hub contoured concavely to engage the thumb and forefinger of a user's first hand, the second grippable member comprises a hub contoured concavel to engage the thumb and forefinger of the user's second hand, and the third grippable member comprises a cylindrical cap with longitudinal ribs adapted to engage the thumb and forefinger of the user's first or second hand.

9. The apparatus of claim 1, wherein the plurality of fiducial carriers comprises three fiducial carriers.

10. The apparatus of claim 1, further including a safety mechanism adapted to prevent unintentional ejection of the fiducials.

11. A method of simultaneously deploying a plurality of fiducials, comprising the steps of:
providing a one-piece apparatus ready to be inserted into an endoscope and capable of simultaneous deployment of a plurality of fiducials, the one-piece apparatus comprising:
an elongated hollow tube fixedly attached with a first grippable member and adapted to be inserted into a patient such that a distal end of the elongated hollow tube is placed proximate a user determined location in the patient
the elongated hollow tube having an interior surrounding a distal portion of each of a plurality of fiducial carriers fixedly attached with a second grippable member longitudinally translatable relative to the first grippable member, each of the plurality of fiducial carriers having a fiducial removably attached therewith near a distal end of the fiducial carrier;
a spreading mechanism adapted to spread the distal ends of the plurality of fiducial carriers, such that at least one of the fiducials is separated from an adjacent fiducial by a fiducial separation distance, when the distal ends of the plurality of fiducial carriers are simultaneously extended beyond the distal end of the elongated hollow tube by longitudinally translating the second grippable member relative to the first grippable member by a predetermined distance; and
an ejecting mechanism adapted to eject and implant the plurality of fiducials simultaneously at a user-determined time after the fiducials are extended beyond the distal end of the elongated hollow tube and separated by the fiducial separation distance, the ejecting mechanism comprising a third grippable member longitudinally translatable and rotatable relative to both the first grippable member and the second grippable member, the third grippable member attached with a plurality of pushers that push the plurality of fiducials out of the fiducial carriers when the third grippable member is longitudinally translated and rotated in a predetermined path relative to both the first grippable member and the second grippable member;
inserting a distal end of the elongated hollow tube to a location in a patient such that the distal end of the elongated hollow tube is placed proximate a user determined location in the patient;
simultaneously extending the distal end of each of the plurality of fiducial carriers beyond the distal end of the elongated hollow tube by longitudinally translating the second grippable member relative to the first grippable member;
spreading the distal ends of the plurality of fiducial carriers, such that at least one of the fiducials is separated from an adjacent fiducial by a predetermined fiducial separation distance by longitudinally translating the second grippable member relative to the first grippable member by a predetermined distance; and
expelling and implanting the plurality of fiducials simultaneously at a user-determined time after the fiducials carriers are extended and the fiducials are separated by the predetermined fiducial separation distance by longitudinally translating and rotating the third grippable member in a predetermined path relative to both the first grippable member and the second grippable member.

12. The method of claim 11, wherein at least a portion of each of the plurality of fiducial carriers are biased to urge away from each other as they are extended beyond the distal end of the elongated hollow tube, and the step of spreading the distal ends of the plurality of fiducial carriers further includes the step of extending said portion of each of the plurality of fiducial carriers beyond the distal end of the elongated hollow tube.

13. The method of claim 11, further including the step of positioning the distal ends of the plurality of fiducial carriers prior to ejecting the plurality of fiducials.

14. The method of claim 11, wherein the steps of expelling and implanting the plurality of fiducials simultaneously when the fiducials are separated comprises simultaneously pushing a plurality of pushers, each of which is at least partially contained in each of the plurality of fiducial carriers and is adapted to eject a fiducial from a corresponding fiducial carrier.

15. The method of claim 14, wherein the translating and rotating movement of the third grippable member relative to both the first grippable member and the second grippable member is limited by a retaining pin that extends from the third grippable member into a keyway in the second grippable member, and wherein the step of simultaneously pushing a plurality of pushers comprises causing the retaining pin to translate through a path defined by the keyway.

16. The method of claim 15, wherein the step of spreading the distal ends of the plurality of fiducial carriers, such that at least one of the fiducials is separated from an adjacent fiducial by a predetermined fiducial separation distance further includes the step of:
spreading the distal ends of the plurality of fiducial carriers, such that at least one of the fiducials is separated from an adjacent fiducial by one or more predetermined fiducial separation distances by pushing the second grippable member distally one or more predetermined hub distances.

17. The method of claim 11, further including the step of operating a safety mechanism adapted to avoid premature ejection of fiducials.

18. A medical targeting system comprising a one-piece apparatus that is ready to be inserted into an endoscope and comprises means for simultaneously deploying a plurality of fiducials in a predetermined spacing.

19. The medical targeting system of claim 18, wherein the means for simultaneously deploying a plurality of fiducials in a predetermined spacing comprises:
first, second, and third gripping means.

20. The medical targeting system of claim 18, adapted to enable user-positioning of a distal end of a fiducial carrier prior to ejecting the plurality of fiducials by movement of the entire one-piece apparatus within an endoscope in a patient.
